(19) United States
(12) Patent Application Publication
(10) Pub. No.: US 2014/0249355 A1
(43) Pub. Date: Sep. 4, 2014

(54) PULSED ELECTROMAGNETIC FIELD DEVICE WITH ADHESIVE APPLICATOR

(71) Applicant: BIOELECTRONICS CORP., Frederick, MD (US)

(72) Inventor: John Robert Martinez, Woodsboro, MD (US)

(73) Assignee: BIOELECTRONICS CORP., Frederick, MD (US)

(21) Appl. No.: 14/352,365

(22) PCT Filed: Oct. 22, 2012
(86) PCT No.: PCT/US2012/061352

§ 371 Date: Apr. 17, 2014

Related U.S. Application Data
(60) Provisional application No. 61/549,645, filed on Oct. 20, 2011.

(51) Int. Cl. A61N 2/00; A61K 36/906; A61M 37/00; A61N 2/02

(52) U.S. Cl. CPC A61N 2/002; A61N 2/02; A61K 36/906; A61M 37/00; A61N 2/004; A61M 2037/0007
USPC 600/14

(57) ABSTRACT

Systems and techniques for applying an electromagnetic field to bodily tissue include a self-contained and portable electromagnetic field generating device adhered to a surface with an adhesive composition on the applicator such that the radiated electromagnetic fields impinge upon the bodily tissue. The adhesive composition may include a therapeutic substance such as a rubefacient and/or one or more additives. The device includes an electromagnetic field generator, which is coupled to an antenna that is arranged to radiate the electromagnetic field. A power source is coupled to the generator to provide power for the device and an activator is used to initiate radiation of the electromagnetic field.

### BACKGROUND

Applying an electromagnetic field to injured bodily tissue has been shown to promote therapeutic healing. In particular, application of a high-frequency electromagnetic field at a sufficiently low field strength so as not to produce tissue heating has been shown to promote a beneficial effect on healing of the tissue. In some cases, effectiveness of the therapeutic effect of the electromagnetic field has been improved by reducing the power requirements of the applied field and extending the treatment duration.

### SUMMARY

The following description relates to an electromagnetic field radiator that influences the metabolic characteristics of living systems. The systems and techniques may be used to therapeutically promote healing of tissue and treat diseases.

In one aspect, a device includes a pulsed electromagnetic field device and an applicator. The pulsed electromagnetic field device includes an electromagnetic field generator, a power source coupled to the electromagnetic field generator, a single-turn loop antenna coupled to the electromagnetic field generator and arranged to radiate an electromagnetic field, and an activator configured to initiate radiation of the electromagnetic field. The applicator includes a flexible substrate defining an opening, an adhesive composition supported by a first surface of the flexible substrate, and a therapeutic substance supported by the first surface of the flexible substrate. The applicator is configured to adhere the pulsed electromagnetic field device to a human body with the adhesive composition such that the therapeutic substance contacts a first portion of the body, a second portion of the body is exposed to air through the opening defined by the flexible substrate, and the radiated electromagnetic field impinges on bodily tissue proximate the first portion and the second portion of the body.

In another general aspect, a device includes a pulsed electromagnetic field device and an applicator. The pulsed electromagnetic field device includes an electromagnetic field generator, a power source coupled to the electromagnetic field generator, a single-turn loop antenna coupled to the electromagnetic field generator and arranged to radiate an electromagnetic field, and an activator configured to initiate radiation of the electromagnetic field. The applicator includes a flexible substrate defining an opening and an adhesive composition supported by a first surface of the flexible substrate. The applicator is configured to adhere the pulsed electromagnetic field device to a surface with the adhesive composition such that a portion of the surface is exposed to air through the opening defined by the flexible substrate. The surface to which the pulsed electromagnetic field device is adhered may be, for example, bodily tissue (e.g., a human body) or an article of clothing intended to be worn close to the surface of the body.

In another general aspect, a kit includes a pulsed electromagnetic field device, an applicator, and instructions for adhering the pulsed electromagnetic field device to a human body with the applicator. The pulsed electromagnetic field device includes an electromagnetic field generator, a power source coupled to the electromagnetic field generator, a single-turn loop antenna coupled to the electromagnetic field generator and arranged to radiate an electromagnetic field, and an activator configured to initiate radiation of the electromagnetic field. The applicator includes a flexible substrate defining an opening, an adhesive composition supported by a first surface of the flexible substrate, a therapeutic substance supported by the first surface of the flexible substrate, and a release liner in contact with the adhesive composition. The instructions describe adhering the pulsed electromagnetic field device to a human body with the applicator such that therapeutic substance contacts a first portion of the body and a second portion of the body is exposed to air through the opening defined by the flexible substrate. The instructions also describe how to initiate radiation of the electromagnetic field. The radiated electromagnetic field impinges on bodily tissue proximate the first portion and the second portion of the body.

Another general aspect includes providing a pulsed electromagnetic field device and an applicator. The applicator includes a flexible substrate defining an opening, an adhesive composition supported by a first surface of the flexible substrate, a therapeutic substance supported by the first surface of the flexible substrate, and a release liner in contact with the adhesive composition. The release liner is removed from the adhesive composition, and the opening defined by the flexible substrate is aligned with an opening defined by the pulsed electromagnetic field device. The pulsed electromagnetic field device is contacted with the adhesive composition supported by the first surface of the flexible substrate, and the pulsed electromagnetic field device is affixed to a human body with the applicator such that the therapeutic substance contacts a first portion of the human body and a second portion of the human body is exposed to air through the opening defined by the flexible substrate. The pulsed electromagnetic field device is caused to radiate an electromagnetic field that impinges on the human body.

Another general aspect includes providing a pulsed electromagnetic field device fixed in an applicator, removing a release liner from the applicator, affixing the applicator to a human body, and causing the pulsed electromagnetic field device to radiate an electromagnetic field that impinges on the human body. The applicator defines an opening and includes an adhesive composition supported by a first surface of the applicator, a therapeutic substance supported by the first surface of the applicator, and a release liner in contact with the adhesive composition. The therapeutic substance contacts a first portion of the human body and a second portion of the human body is exposed to air through the opening defined by the applicator.

Another general aspect includes positioning a pulsed electromagnetic field device and an applicator proximate a human body, affixing the pulsed electromagnetic field device to the human body with the applicator such that the therapeutic substance contacts a first portion of the human body and a second portion of the human body is exposed to air through the opening defined by the flexible substrate, and causing the pulsed electromagnetic field device to radiate an electromagnetic field that impinges on the human body. The applicator includes a flexible substrate defining an opening, an adhesive composition supported by a first surface of the flexible substrate, and a therapeutic substance supported by the first surface of the flexible substrate.

Another general aspect includes positioning a pulsed electromagnetic field device fixed in an applicator proximate a human body, affixing the applicator to the human body such that the therapeutic substance contacts a first portion of the human body and a second portion of the human body is exposed to air through the opening defined by the flexible substrate, and causing the pulsed electromagnetic field device to radiate an electromagnetic field that impinges on the human body. The applicator includes a flexible substrate defining an opening, an adhesive composition supported by a first surface of the flexible substrate, and a therapeutic substance supported by the first surface of the flexible substrate.

Another general aspect includes adhering a pulsed electromagnetic field device to an article of clothing with an applicator, causing the pulsed electromagnetic field device to radiate an electromagnetic field, and dressing a human body with the article of clothing, such that the pulsed electromagnetic field device is proximate the human body and the radiated electromagnetic field impinges on the human body.

Implementations of all of the above general aspects may include one or more of the following features as applicable. In some cases, a release liner is in contact with the adhesive composition. The adhesive may be non-toxic and/or hypoallergenic and/or FDA-approved. For example, the adhesive can be a pharmaceutical grade adhesive. In some cases, the adhesive composition includes the therapeutic substance. The adhesive composition may also include one or more additives, such as an antibacterial or antimicrobial additive, a hydrogel, etc. In certain cases, the therapeutic substance and/or one or more additives may be applied as separate layers on the flexible substrate rather than (or together with) being mixed in with the adhesive composition.

The therapeutic substance may be a topical rubefacient. The rubefacient may include, for example, a salicylate, a nicotinate ester, capsaicin, isopropanol, menthol, or a combination thereof. In some cases, the rubefacient includes an extract or oil of cloves, garlic, ginger, horseradish, mustard, nettle, rosemary, rue, or a combination thereof. The therapeutic substance may have analgesic properties, anti-inflammatory properties, anesthetic properties, or a combination thereof. In certain cases, the duration of the electromagnetic field radiation exceeds the duration of efficacy of the therapeutic substance (i.e., the pulsed electromagnetic field device is still operating after the effects of the therapeutic substance have worn off).

In another aspect, a device for applying a therapeutic electromagnetic field is disclosed including an electromagnetic field generator, which is coupled to an antenna that is arranged to radiate the electromagnetic field. A power source is coupled to the generator to provide power for the device and an activator is used to initiate radiation of the electromagnetic field. The therapeutic device is self-contained and portable and is disposed over a surface of bodily tissue such that the radiated electromagnetic field impinges upon the bodily tissue.

In an implementation, the power source is a battery of less than approximately 10 VDC. In another implementation, the device is a component of a therapeutic delivery system. The therapeutic delivery system includes a member from the group of a patch, a bandage, a pad, a brace, a strap, tape, adhesive and a cast.

In another aspect, a technique for applying a therapeutic electromagnetic field is facilitated by incorporating a power source, antenna and electromagnetic field generator within a portable and disposable package and affixing the device to bodily tissue. The device generates an electromagnetic field that induces an alternating current in the bodily tissue. In another implementation, the average available radiated power is less than approximately 1 milliwatt and the peak available radiated power density is less than 100 microwatts per square centimeter measured substantially at the surface of the tissue.

Some implementations of the systems and techniques described herein may provide one or more of the following advantages. The device may be suitable for prolonged use. The self-contained unit can encourage patient compliance. In some implementations the device may be placed directly over bodily tissue to provide electromagnetic therapy to the tissue. The device may be part of a therapeutic agent delivery system such as a patch, bandage, pad, brace, cast, or other tissue injury support device.

In another aspect, a method is disclosed for inducing electrical current in a bodily tissue by: (1) positioning a device described herein adjacent a bodily tissue of an individual; and (2) operating the device for a duration, at a frequency, and at a peak available radiated power density effective to induce electrical current in the bodily tissue, wherein the device is positioned relative to the individual such that the device induces electrical current in the bodily tissue without making conductive contact with the bodily tissue. In some embodiments, the induction of electrical current in the bodily tissue reduces or eliminates a pain sensation in the individual.

In another aspect, a method is disclosed for treating an individual by: (1) positioning a device described herein adjacent a bodily tissue of an individual; and (2) operating the device for a duration, at a frequency, and at a peak available radiated power density effective to elicit a therapeutic response in the individual, wherein the device is positioned relative to the individual such that the device induces electrical current in a bodily tissue of the individual without making conductive contact with the bodily tissue.

In another aspect, a method is disclosed for treating an individual by: (1) providing a device containing an electromagnetic field generator; (2) positioning the device adjacent a bodily tissue of an individual; and (3) operating the device for a duration, at a frequency, and at a peak available radiated power density effective to elicit a therapeutic response in the individual, wherein the device is positioned relative to the individual such that the device induces electrical current in the bodily tissue of the individual without making conductive contact with the bodily tissue, and wherein the device effects a penetration of the induced current into the bodily tissue such that the therapeutic response is elicited at a depth of at least 2 cm in the bodily tissue. In some embodiments, the therapeutic response is elicited at a depth of at least 3, 4, 5, or 6 cm in the bodily tissue. In other embodiments, the therapeutic response is elicited at a depth of 2 to 3, 2 to 4, 2 to 5, 2 to 6, 3 to 4, 3 to 5, or 3 to 6 cm in the bodily tissue.

In another aspect, a method is disclosed for treatment by: (1) providing a device selected from the group consisting of a pulsed electromagnetic field therapy (PEMF) apparatus, a transcutaneous electrical neural stimulator, and a static magnet array; (2) positioning the device at a distance from an individual effective to elicit a therapeutic response in the individual, wherein the device is positioned at a bodily location selected from the group consisting of the external end of the elbow transverse crease, the depression at the lower border of the malleolus lateralis, below the lateral extremity of the clavicle at the level of the first intercostals space, between the fourth lumbar vertebra and the fifth lumbar vertebra or 1 inch to the right or left thereof horizontally, a depression anterior or inferior to the head of the fibula, about 1.5 inches above the medial border of the patella, and between the radius and the palmaris longus; and (3) maintaining the device at the bodily location for a duration effective to elicit the therapeutic response.

In the methods described herein, positioning a device adjacent a bodily tissue of an individual refers to placing the device close to the skin of the individual (e.g., within 0.5, 1, 2, 3, 4, 5, or 6 inches of the skin) or in contact with the skin. The device can be encapsulated in a material and still be considered adjacent a bodily tissue, so long as it operates in the manner described herein. The methods do not entail penetration of the skin by the device and/or the application of electrodes to the skin (e.g., the device induces current in a bodily tissue in the absence of an application of electrodes to the skin). Tissues that can receive the electrical current according to the methods described herein include, for example, the skin as well as tissues that underlay the skin (e.g., joints or bones).

An exemplary device for use in the methods described herein comprises: an electromagnetic field generator; an antenna coupled to the generator and arranged to radiate the electromagnetic field; a power source (e.g., a battery) coupled to the generator; and an activator to initiate radiation of the electromagnetic field, wherein the device is self-contained and portable. The antenna can optionally contain antenna conductors on a printed circuit board. In some embodiments, the device additionally contains: an annular ring to surround the battery; and a wire wound around the annular ring. In some embodiments, the annular ring has a stepped cross-section and a wire wound on a top and outer side of the annular ring coupled to the antenna conductors. In some embodiments, the annular ring contains a ferrite ring. In some embodiments, the annular ring contains an insulating-magnetic ring.

The current induced in the bodily tissue of an individual can be, for example, parallel or perpendicular to the direction of antenna conductors.

In some embodiments of the methods described herein, the frequency is 27+/−0.5 MHz (e.g., 27.1 MHz).

In some embodiments of the methods described herein, the peak available radiated power density is less than 100 microwatts per square centimeter measured at the surface of the bodily tissue (e.g., the skin of the individual).

The device used in the methods can optionally contain a delivery system, e.g., a patch, bandage, pad, brace, strap, tape, adhesive, or cast. In some embodiments the delivery system is a single use adhesive bandage.

The methods described herein can additionally include pulsing the generated electromagnetic field. In addition, the methods can also include altering at least one of a duty-cycle and a pulse repetition rate of the pulsed electromagnetic field. In some embodiments, the duty cycle is approximately 8%-10%.

In some embodiments, the individual has a pain-related disorder and the therapeutic response includes a reduction or elimination of pain in the individual. Examples of pain-related disorders include, for example, pain response elicited during tissue injury (e.g., inflammation, infection, and ischemia), pain associated with musculoskeletal disorders (e.g., joint pain such as that associated with arthritis, toothache, and headaches), pain associated with surgery, pain related to irritable bowel syndrome, and chest pain.

In some embodiments, the individual has a disorder selected from the group consisting of adhesive capsulitis, tennis elbow, osteoarthritis, back pain, multiple sclerosis, tendon inflammation, and carpal tunnel syndrome, and the therapeutic response includes a reduction or elimination of pain associated with the disorder.

In some embodiments, the individual has a bone, joint, soft-tissue, or connective tissue disorder and the therapeutic response includes a reduction or elimination of inflammation in a bone, joint, soft-tissue, or connective tissue of the individual. In some embodiments, the individual has a bone, joint, soft-tissue, or connective tissue disorder and the therapeutic response includes a reduction or elimination of pain associated with the disorder.

In some embodiments, the individual has a dental condition, and the therapeutic response includes a reduction or elimination of pain associated with the condition.

In some embodiments, the individual has an arthritic disorder and the therapeutic response includes a reduction or elimination of pain associated with the disorder. In an example, the disorder is osteoarthritis of the knee and the therapeutic response includes a reduction or elimination of pain of the knee.

Details of one or more implementations are set forth in the accompanying drawings and the description below. Other features and advantages will be apparent from the description and drawings, and from the claims.

### BRIEF DESCRIPTION OF THE DRAWINGS

FIG. 1 is an implementation of a therapeutic electromagnetic device depicting an arrangement of the components.

FIG. 2 is an implementation of a therapeutic electromagnetic patch depicting components in layers.

FIG. 3 is a block diagram of an implementation of a therapeutic electromagnetic device.

FIGS. 4A-B illustrate a control waveform and resulting RF waveform.

FIGS. 5A-I illustrate antenna configurations.

FIG. 6 depicts an annular electromagnetic device adhered to bodily tissue.

FIGS. 7A-D depict various applications of a PEMF device.

FIGS. 8 and 8A depict an enhanced antenna.

FIG. 9 depicts anatomical locations for placement of a therapeutic device.

FIG. 10A depicts a PEMF device with a single-turn loop antenna.

FIG. 10B depicts an applicator for adhering the PEMF device of FIG. 10A.

FIG. 10C shows the PEMF device of FIG. 10A adhered to the applicator of FIG. 10B.

FIG. 10D depicts the PEMF device of FIG. 10A adhered to a human body with the applicator of FIG. 10B.

FIG. 10E is a side view of the applicator shown in FIG. 10C aligned above the PEMF device of FIG. 10A.

FIG. 10F depicts the applicator of FIG. 10C in contact with the PEMF device of FIG. 10A.

FIG. 10G depicts the PEMF device of FIG. 10A adhered to a surface with the applicator of FIG. 10C.

FIG. 10H depicts the PEMF device of FIGS. 10A and 10B fixed in an applicator prior to application to a surface.

FIG. 10I depicts the PEMF device of FIG. 10H adhered to a surface.

FIG. 11A depicts a front view of a PEMF device with a single-turn loop antenna.

FIG. 11B depicts the PEMF device of FIG. 11A adhered to an applicator prior to application to a surface.

FIG. 12A depicts a kit including a PEMF device, an applicator, and instructions for use.

FIG. 12B depicts a kit including a PEMF device fixed in an applicator and instructions for use.

FIGS. 13A-13E are flow charts describing application of a PEMF device to a surface with an applicator.

Like reference symbols in the various drawings indicate like elements.

### DETAILED DESCRIPTION

The systems and techniques described here relate to promoting therapeutic healing of tissue, providing prophylaxis for, and treatment of disorders and diseases through the application of an electromagnetic field. The techniques include providing a self-contained miniaturized electromagnetic field generating device that may be applied to bodily tissue (e.g., to a human body). In some implementations the techniques and systems include devices that are disposable and portable.

The generated electromagnetic field can induce alternating current in bodily tissue. The alternating current may be subjected to non-linear electrical characteristics (for example, diode-like rectification) and so generate low frequency electrical potentials having a time dependence the same as the pulse modulation. The low frequency electrical potentials may stimulate cellular communication by, for example, altering the frequency of cellular activation potentials. Cellular communication may promote the healing of inflammation and the reduction of edema.

These techniques also may provide a method of transmission and utilization of the body's capacitance by affixing a transmitting element of the device to conform and fit closely over the bodily tissue, provide a small space and low weight device for field transport and emergency use. Patient compliance with a therapeutic regimen may be important to promote healing of bodily tissue. Patient compliance may be improved by providing a therapeutic device that is self-contained and portable.

Some or all of the components of a therapeutic electromagnetic energy delivery device may be integrated into a control circuit chip to miniaturize the device. The device may be affixed to various parts of the body for prolonged electromagnetic therapy. Patient compliance to the therapeutic regimen may be improved by embedding or concealing the device into a patch, bandage, pad, wrap, brace, cast, or other injury support device and affixed to the body or taped over the bodily tissue.

The effectiveness of electromagnetic therapy may be improved by extending the treatment duration. Lower power electromagnetic radiation may be applied for a longer period of time than may be necessary for shorter periods of application. The self-contained unit disclosed may promote patient compliance with periods of therapy that may extend over weeks.

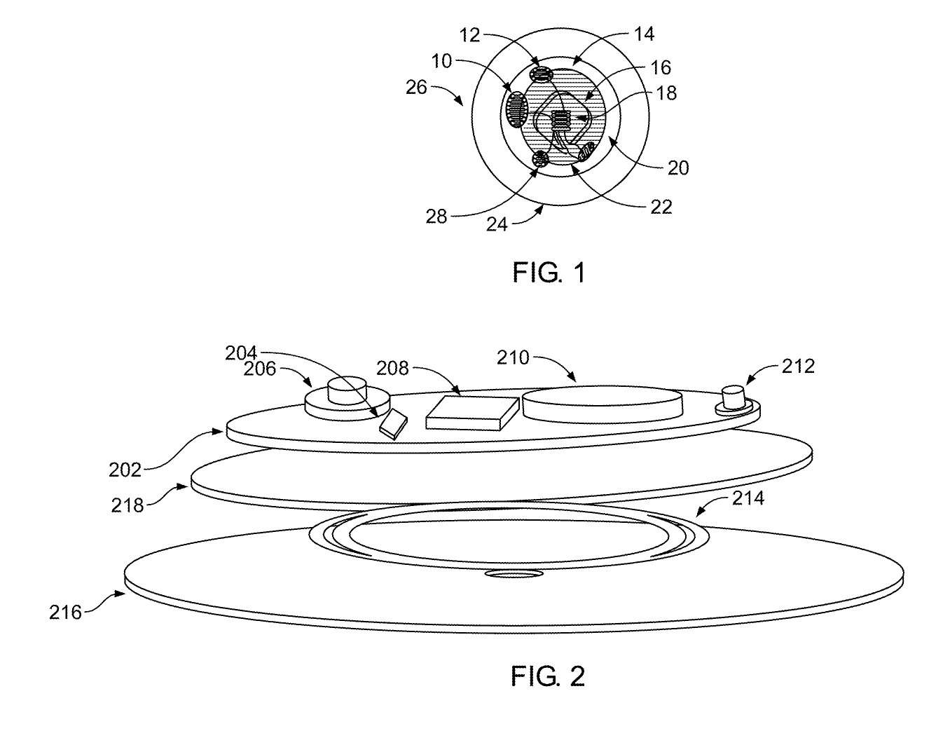

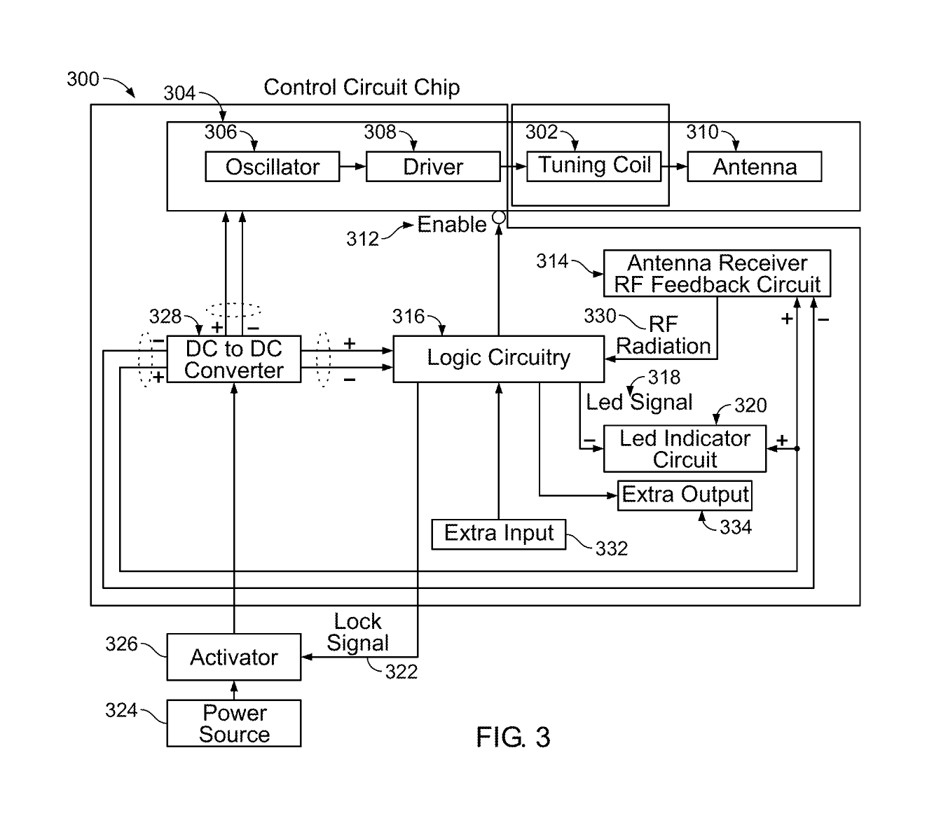

FIG. 1 illustrates an implementation of a therapeutic electromagnetic device 26. A control circuit chip 18 may provide the functionality for the therapeutic electromagnetic device to operate. An implementation of a control chip 18 is disclosed in association with the description of FIG. 3 and includes a radio frequency (RF) generator. A power source 10 coupled directly or indirectly to the control chip may be used to power the therapeutic electromagnetic device. The power source may include a battery, photovoltaic cell or an electro-chemical cell. An activator 12 is used to activate the device. The activator may include a switch that is a single-use or multiple use type and may be momentary or alternate-action. Actuation of the activator may be accomplished in various ways including by use of pressure, light or electronic signal either remotely or proximately. An antenna 16 is used to emit electromagnetic radiation and a deflector shield 14 may be used to deflect the electromagnetic radiation to the bodily tissue. In an implementation, the antenna 16 and/or deflector 14 may be tuned for electromagnetic energy in the frequency range of 27±0.5 Mhz. The therapeutic electromagnetic device also may include a tuning coil 20 which may be used to match the impedance of the antenna 16 to the RF signal generator within the control circuit chip 18. A circuit board 22 may be used to mount the elements of the device and, in some cases, provide coupling between the elements of the device. The circuit board may be comprised of a rigid or flexible material. The assembled device weighs less than 12 grams.

In some implementations, an adhesive material 24 may be used for affixing the therapeutic electromagnetic device to bodily tissue. Adhesive material 24 includes, for example, pharmaceutical grade adhesives. The therapeutic electromagnetic device may be affixed using other single or multiple usage therapeutic delivery devices, which include a patch, a bandage, a pad, a brace, a strap, tape, adhesive and a cast. In some implementations, an indicator 28 can be used to provide indicia that the therapeutic electromagnetic device is active. The indicator 28 may include one or more of the following: a visual indicator such as a light emitting diode (LED), lamp or electro-luminescent display; an auditory indicator such as noise generator; or a tactile indicator such as a vibrator. In an implementation, the indicator may be coupled to an electromagnetic field detector in the control circuit chip 18 and indicate the presence or lack of electromagnetic radiation from the device. In various implementations the indicator may be steady, intermittent or pulsed.

The therapeutic electromagnetic device may be enclosed or encapsulated in encapsulants or other potting compounds to reduce the vulnerability of the device to foreign materials including moisture, fluids, fungus, static charges, dirt, particulate matter and dust. The encapsulants, including insulating resins such as epoxies, polyurethanes, and polyesters, may be cast into cavities containing the device components, to insulate, protect, and hold the components in place. The encapsulant also may reduce the vulnerability of the device to environmental factors including air, heat, sunlight, ultraviolet light and spurious electromagnetic fields. In some implementations, a conformal coating may be applied to the device components and couplings to reduce the vulnerability of the device to moisture, fluids, fungus, static charges, dirt, particulate matter and dust.

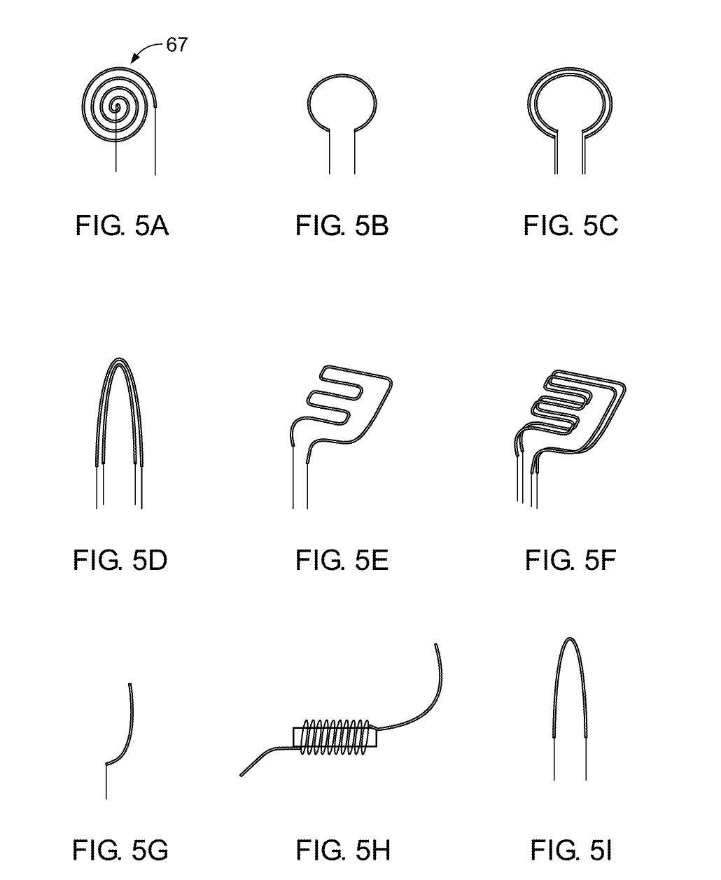

FIG. 2 illustrates an exploded view of an implementation of the therapeutic electromagnetic device having the components in a layered form. An activation switch 206, a control circuit chip 208, a power source 210, a visual indicator 212 and a tuning coil 204 may be mounted on a top layer and attached to a circuit board 202 to provide coupling between the components. A deflecting shield 218 may be layered under the circuit board 202. Deflecting shield 218 may be a layer or coating of material, having high magnetic permeability, applied directly to circuit board 202. An antenna 214 to radiate electromagnetic energy may be layered under deflecting shield 218 and coupled to the circuit board 202. The deflecting shield 218 may deflect some of the energy radiated from the antenna 214 away from components mounted on the circuit board and toward the bodily tissue. The shape of the antenna is not restricted and some common shapes are depicted in FIGS. 5A-I. The antenna may also include separate conductors that do not make electrical contact with each other. In some implementations, the antenna may have a thickness of less than 5 millimeters and diameter of less than 9 centimeters or in other implementations, a length of less than 27 centimeters. The antenna may be incorporated into the circuit board 202.

The shape of the circuit board 202 and deflecting shield 218 may be altered to adapt the therapeutic device to particular applications. The thickness of the device is less than 10 millimeters. In one implementation, an adhesive material 216 such as a pharmaceutical adhesive may be mounted to the bottom layer under antenna 214 to adhere the device to bodily tissue. Other therapeutic delivery devices including a patch, a bandage, a pad, a brace, a strap, tape, adhesive and a cast also may be used. In some implementations, the components may be selected and arranged for specific applications.

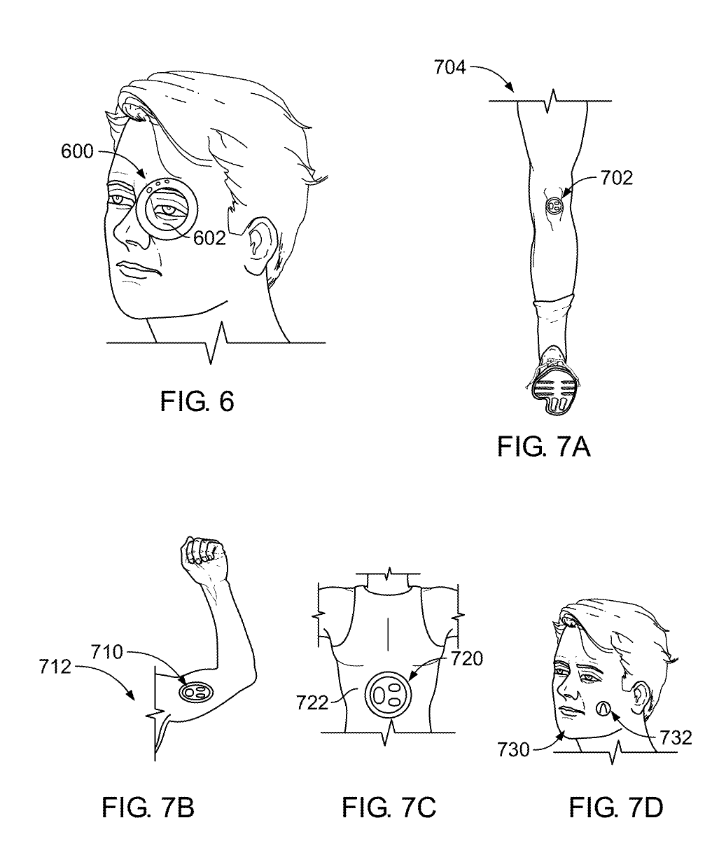

Referring to FIG. 6, for example, the therapeutic device 600 may have a generally annular shape in a therapeutic application such as post-operative healing over an eye or breast. In this case, the annular shape defines a hole 602 through which a patient may see while the device is in place.

FIG. 3 is a block diagram of the circuitry of one implementation of a control circuit chip 300 used in a therapeutic electromagnetic device. Optionally, a tuning coil 302 may be included within the control circuit chip 300 or mounted separately. The components of the control circuit chip 300 may be integrated into one part or may be assembled from discrete components. The control circuit chip 300 includes an electromagnetic field generator 304 comprised of an oscillator 306 and a driver 308. Logic circuitry 316 coupled to the generator 304 provides an enable signal 312 to the generator 304. The logic circuitry also may provide an LED signal 318 to an indicator circuit 320, which, in turn, may be coupled to an indicator (not shown). Logic circuitry 316 may include discrete components, a programmable logic device (PLD), a microprocessor or other micro-controller unit (MCU). A power source 324 may be used to supply power to the electromagnetic therapy device. An activator 326 controls the flow of power from the power source to a DC to DC converter 328. The activator includes a switch that can provide for a one-time activation and then sustain activation for the duration of life of the power source. The DC to DC converter 328 provides power to the control chip components including the logic circuitry 316, the electromagnetic field generator 304 and an optional RF feedback circuit 314. The RF feedback circuit provides an RF radiation signal 330 to the logic circuitry 316. The logic circuitry also may provide an LED signal 318 to an LED indicator circuit and a lock signal 322 to the activator 326.

The electromagnetic field generator 304 comprises an oscillator 306 to generate an electromagnetic field, a driver circuit 308 to receive the electromagnetic field, amplify the wave and to provide the amplified wave to the optional tuning coil 302. The tuning coil 302 may be used to match the impedance of the driver 308 to an antenna 310, which is arranged to radiate the amplified electromagnetic energy. The oscillator 306 may be arranged to produce electromagnetic waves, including sinusoidal waves, at a carrier frequency of 27+/−0.5 megahertz (MHz). In an implementation, the electromagnetic therapeutic device has an average available power of less than approximately 1 milliwatt and a peak available radiated power density of less than 100 microwatts per square centimeter (μW/cm²) measured substantially at the surface of the tissue. The electrical efficiency of average available radiated power generation also may be greater than 20%. Average available power is the power that the device can dissipate into a resistive load. The average available power is distinguished from the power of the carrier within each pulse, which is termed the “peak” power. The peak available radiated power density is the maximum carrier wave power as if it was continuous and not pulsed, divided by the loop area of the antenna. A high voltage generator (not shown) may be included to increase the intensity of the radiated field. The high voltage generator may produce less than 30 VDC and may be synchronized to allow energy transforming action between therapy pulses, so that therapy pulses are not affected by the energy transformation action. Energy transformation could comprise connecting the battery to an inductive coil for a brief duration, and then switching the coil into a diode or rectifier and capacitor. The capacitor accumulates charge at a higher voltage than the battery. When voltage on the capacitor reaches a predetermined value, the capacitor may be discharged into the frequency generator for producing a therapy pulse. Alternatively, a transformer connected to a rectifier and capacitor as a flyback transformer may replace the inductive coil.

The enable signal 312 may be used to initiate or curtail radiation of the electromagnetic energy. The RF feedback circuit 314 is arranged to detect RF radiation from the antenna 310 and to provide RF radiation signal 330 to logic circuitry 316. Based on the level of the RF radiation signal 330, the logic circuitry provides the LED signal 318 to enable/disable the LED indicator circuit 320, which drives the indicator (not shown) and provides an indication that the antenna is radiating electromagnetic energy. The logic circuitry 316, the LED indicator circuit 320 or the indicator may be arranged so that the indicator is either indicating continuously, intermittently or pulsating. The logic circuitry also may provide the enable signal 312 to enable/disable the electromagnetic field generator 304.

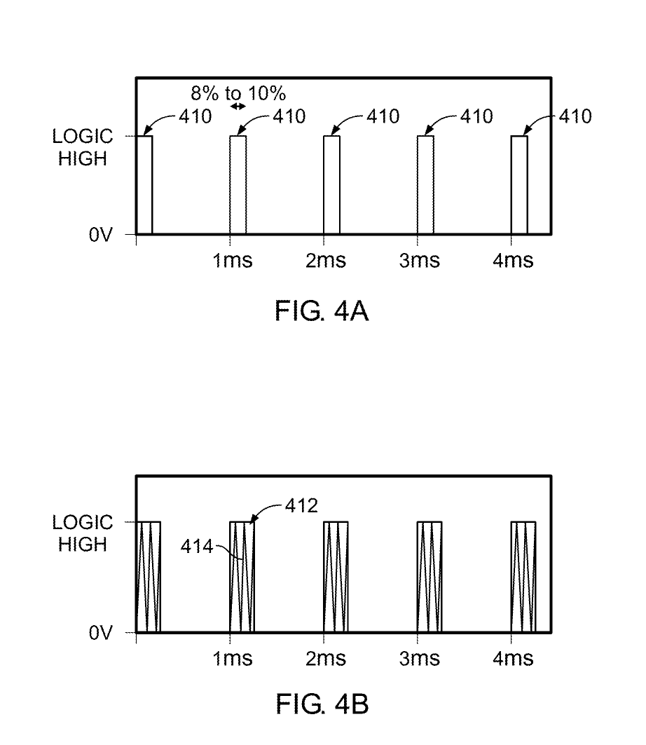

In an embodiment, the energy radiated by the antenna 310 may be pulsed. A PEMF device may be used to provide electromagnetic field therapy over long periods of time and reduce heating of the bodily tissue. FIG. 4A illustrates that an enable signal 410 that may be provided from the logic circuit 316 to enable the generation and radiation of electromagnetic energy. In this example, the enable signal goes to a logic level high every millisecond. The enable pulse level is shown as a logic high but alternatively may be a logic low. In some implementations, the logic high level may be the power source, or regulated non-zero, voltage although other voltages are possible. The illustrated duty cycle is approximately 8% to 10%. In some implementations, the electromagnetic therapeutic device may operate in the frequency range of 3-30 MHz and application of the electromagnetic energy may be pulsed to maximize the therapeutic effect of the field. Pulses of 100 microsecond (μS) pulse duration at intervals of 1 millisecond (mS) (a pulse repetition rate of 1000 Hz) may be preferable. In order to reduce heating of the tissue, the electromagnetic field strength may be limited to less than 100 micro-Watts per square centimeter (μWcm⁻²) as measured proximate the surface of the tissue. FIG. 4B illustrates a resulting output 412 from the antenna. The electromagnetic field 414 is radiated from the antenna only when the enable signal 410 is at a logic high.

Referring again to FIG. 3, the power source 324 may be direct current (DC) and preferably less than approximately 10 VDC. The power source may be rechargeable. The rechargeable power source may be a battery of the lithium metal hydride or lithium ion or lithium polymer technology that may be recharged from an external source, including a sine wave field generator proximate the antenna 310 or separate coil (not shown) for the non-contacting induction of power from the external source into the therapeutic device. Current induced in the antenna or separate coil may be rectified and supplied as a reverse current to the rechargeable power source until the power source reaches a predetermined terminal voltage or case temperature.

The power source 324 is coupled to the activator 326. When the activator is actuated, power is coupled to the DC to DC converter which may boost and regulate the power source voltage level. Regulated output voltage from the DC to DC converter 328 is supplied to the logic circuitry 316, electromagnetic field generator 304 and RF feedback circuit 314. A lock signal 322 may be provided by the logic circuitry 316 to lock the activator in the “on” position when the activator is actuated at least once. Optionally, extra input signals 332 and extra output signals 334 may be received and/or provided by the logic circuitry 316 for additional functionality. For example, an output signal may be provided that provides indicia of the level of the voltage level of the power source 324. The output signal may activate a visual or auditory alarm when the power source requires replacement. An output signal may be provided that provides indicia of a state of the bodily tissue. The electrical permittivity and conductivity of tissue affects the frequency of the carrier wave in the device. The ratio of conductivity (σ) to permittivity multiplied by angular frequency (ωε) determines the polarity of the frequency change. If σ exceeds ωε then the carrier frequency decreases. If ωε exceeds σ then the carrier frequency increases. As conductivity is related to pH and free ion concentration, while permittivity is related to abundance of polar molecules and cell membrane charge, the bioelectrical state of the tissue may be assessed by determining the carrier frequency change from that at initial application of the device.

Optionally, the extra output signal 334 may provide control by enhancing the electromagnetic field for directed movement of chemical or pharmaceutical molecules in tissue, such as silver ions, for infection control. The enhanced electromagnetic field may be non-uniform in such a way as to direct movement of polar molecules, a method known as dielectrophoresis. Alternatively, the enhanced electromagnetic field may induce an electric field, which directs the movement of ions, a method known as iontophoresis.

An input 332 may be provided to receive external signals, for example, that alter the electromagnetic pulse duration, duty-cycle or pulse repetition rate of the electromagnetic field generated.

FIGS. 7A-D depict some applications of the therapeutic electromagnetic device. FIG. 7A depicts a therapeutic electromagnetic device affixed to a knee of a human leg 702. The device may be applied to aid in healing of, for example, a cracked knee, a cut, a sprain or strain. FIG. 7B depicts a therapeutic electromagnetic device 710 affixed to a muscle of a human arm 712 to aid in the healing of, for example, a sprain, a strain or a cut. FIG. 7C depicts a therapeutic electromagnetic device 720 affixed to a human abdomen 722 where, for example, lipo-suction procedures were performed. FIG. 7D depicts a human face 730 where a therapeutic electromagnetic device 732 is affixed on a left side of the face to aid in healing of an injury such as a tooth cavity.

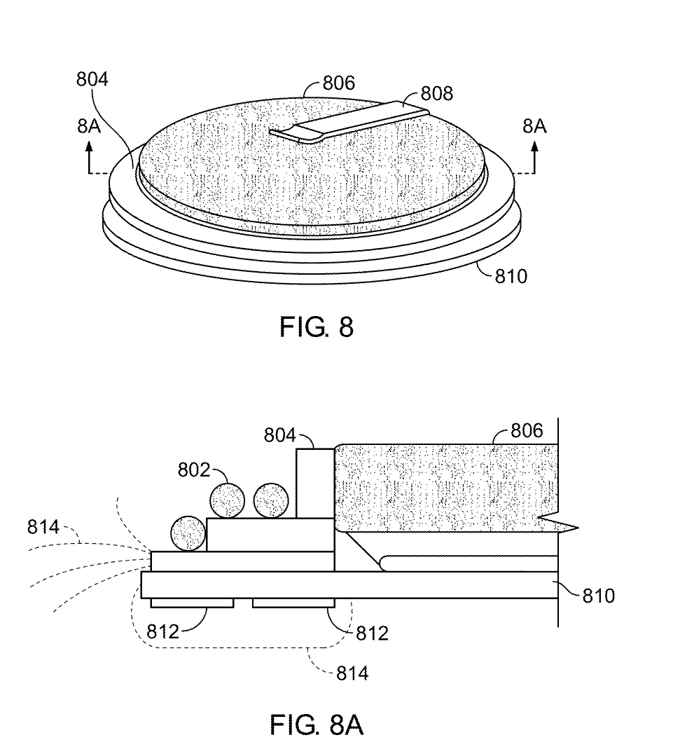

FIG. 8 depicts an implementation of an enhanced antenna comprising wires 802 wound around an annular ring 804 mounted on a printed circuit board 810. The ring may be a ferrite or magnetic, electrically-insulating ring. The ring may be arranged to support a battery 806 around the periphery. The battery 806 may be held in place by a retaining clip 808 to retain the battery adjacent the printed circuit board 810. Conductors 812 on the printed circuit board may be arranged to function as a main antenna for the therapeutic electromagnetic device and may be coupled to an electromagnetic field generator (not shown) as described above.

The annular turns of the wires 802 can convey current in phase and frequency with the main antenna 812. The number of turns of wire 802 on the annular ring are arranged to provide a larger magnetic flux than that of the main antenna 812. The windings cause a magnetic flux to enter/exit the outer perimeter of the annular ring. A portion of the (alternating) flux impinges bodily tissue underneath the therapeutic electromagnetic device inducing additional alternating current concentric with the main antenna. The additional induced current may result in magnetic flux that could otherwise be generated by a main antenna having a larger diameter. The magnetic field lines 814 from the main antenna conductors on the printed circuit board will take the path of least magnetic reluctance and pass around the underside of the printed circuit board. Only a weak magnetic field impinges the battery 806. The larger portion of the field may be restrained near the main antenna conductors. The effect is to generate increased magnetic field intensity farther in the bodily tissue. Thus, the main antenna, such as a simple or single-turn loop antenna, with the enhanced antenna windings on the annular ring can present as an antenna with a larger effective diameter.

A simple loop antenna can produce a near field of electromagnetism, which can be confined within a certain volume by the physical geometry of the antenna. The magnetic field on the axis of a circular loop antenna diminishes in proportion to:

$$MagneticField \approx \frac{1}{{( 1 + {( \frac{z}{a} )}^{2} )}^{1.5}}$$

where z is the distance from the center of the loop and a is the radius of the loop. Beyond a distance Z, the current induced by the magnetic field in the bodily tissue may be ineffective to provide therapeutic value. The distance Z is measured at the point where the surface of the volume intersects the axis. A therapy volume wherein the electromagnetic field induced in the bodily tissue is adequate to have therapeutic value can be determined from the radius, and circularity, of the loop antenna and the current flowing in the antenna. Outside of this volume, therapy may be inadequate. Inside this volume, therapy may be effective and diminishing on approach to the surface of the therapy volume. In some embodiments, the device effects a penetration of induced current into the bodily tissue such that a therapeutic response is elicited at a depth of at least 2 cm in the bodily tissue.

A larger effective diameter antenna can increase the magnitude of the induced current and extend the depth of penetration of induced current. Hence, the main antenna with the enhanced antenna may result in current induction inside the bodily tissue over a larger area and to a greater depth than with the main antenna alone.

### Method of Using Pulsed Electromagnetic Field (PEMF) Therapy in Certain Diseases

Bone and Joint Disorders: The urine of patients with bone and joint disorders typically shows elevated levels of hydroxyproline, hexosamine, creatinine, and uronic acid as a result of metabolic errors in connective tissues surrounding the affected site. Not only can these errors be corrected with PEMF therapy, but joint pain and swelling can be reduced and mobility of the joint increased. Another major advantage of PEMF therapy is that it significantly reduces the time required to heal fractured bones. It has also proven to be effective for osteomyelitis, osteoarthritis, rheumatoid arthritis, cervical spondylosis, and lower back pain (including that caused by disc displacement).

Diabetes Mellitus: Blood sugar levels may be slowly reduced to normal or near normal with application of a pulsed electromagnetic field (PEMF). Although the mechanism of action is not completely understood, the evidence obtained thus far indicates that the procedure not only increases the metabolism of glucose in the tissues but also increases the production of insulin and enhances insulin binding to its specific receptors. The therapy has also proven to be effective for gastritis, peptic ulcer, ulcerative colitis, irritable colon, and hemorrhoids.

Bronchial Asthma: Bronchiolar obstruction can be gradually reduced with PEMF treatment, which liquifies the mucous and facilitates spontaneous clearance. PEMF therapy also has anti-inflammatory action, which helps to ensure that the airways remain free and functional. In patients who have undergone the treatment, Forced Vital Capacity, Forced Expiratory Volume, and Peak Expiratory Flow Rates have increased and wheezing and dyspnea have significantly improved. The treatment is also effective for the common cold, tonsillitis, sinusitis, chronic bronchitis, bronchiectasis

Cardiovascular Diseases: PEMF therapy is useful in the prevention of heart attacks in hypertensive patients. Treatment helps to lower blood cholesterol levels and increase the circulation of blood by centrally mediating vascular dilatation. This is particularly important in preventing platelet aggregation and maintaining adequate oxygenation and nutrition of cardiovascular and other tissues. PEMF therapy also effectively disintegrates atherosclerotic plaques. An additional advantage of the procedure is that it blocks the production of free radicals, which play a major role in cardiovascular damage at the cellular level. Other vascular conditions for which PEMF may be effective are phlebitis, endarteritis, and varicose vein.

Brain and Mind Disorders: Directed through the skull at different points, the PEMF can, by inductive coupling, produce an electric current in specific areas of the brain. It may thus be possible to enhance higher brain functions such as learning, memory, and creative thinking by selective stimulation of certain cells. PEMF may have broad application as the modality of choice for psychological disorders such as depression, aggression, anxiety, and stress as well as for Parkinson's disease, epilepsy, migraine, stroke, Alzheimer's and other degenerative brain disorders. In addition, cerebral palsy, mental retardation, hyperactivity, learning disabilities may be improved by PEMF stimulation of the central nervous system.

PEMF therapy can increase the efficiency of brain cells in synthesizing the neuro-chemicals required for the transmission of impulses or commands at the synaptic level and by improving the electrical activity of these cells. The brain is a neuro-chemical complex. The efficiency of the brain or intellectual capacity of the brain depends upon the efficient performance of the brain cells and production of the chemicals that are called neurotransmitters.

Too much dopamine can result in hyperactivity, while too little can result in uncoordinated movements of the limbs (Parkinsonism). Less acetylcholine, a neuro-chemical, in the brain is a reason for dementia especially of the Alzheimer's type. If the brain cells are stimulated repeatedly, after showing inhibition, they rebound and become more active than prior to stimulation. Since PEMF has the ability to stabilize the genes and prevent the activity of oxygen free radicals formed in the cells, it helps to retard the aging process.

Genitourinary Conditions: PEMF has been successfully used to treat genitourinary conditions such as menstrual irregularity, sterility, endometritis, and endometriosis in women and orchitis, prostatitis, and oligospermia in men.

Preoperative and Prophylactic Therapy: PEMF therapy over the epigastrium can provide increased blood profusion to the body's extremities to reduce the inflammatory response to injury. Preoperative treatment of the surgical site has also been shown to accelerate healing.

Post-Operative Recovery: PEMF or TENS over 1.5 inches above the wrist line may reduce or ease the nausea for post-surgical recovery, motion sickness or other forms of nausea symptoms such as vomiting.

Non-Contacting Induction of Electrical Current in Tissue

Devices described herein can induce current at a high frequency. The amount of current induced by a device is partly proportional to the frequency. Modulating a carrier waveform, such as the pulse modulation of 27+/−0.5 Mhz (e.g., 27.1 MHz) in devices described herein, allows a larger current to be produced in a tissue than the pulse modulation waveform alone. The pulse modulation is selected for time and amplitude characteristics appropriate to biological systems. The carrier wave ensures that induced current has a magnitude that is maintained coherently within the pulse modulation. A varying pulse modulation is sustained by a similar magnitude of induced current. Rectification occurring in biological systems, such as across cellular membranes, causes the originating pulse modulation waveform to appear as a low frequency voltage. Membrane capacitance allows induced currents to enter cells much more easily than the pulse modulation waveform would by itself. Shunting of current around cells rather than through the cells is also reduced.

No conductive contact of the device with the tissue is required to induce the electrical current in the tissue. The size of the antenna of the device, being much smaller than a wavelength, ensures that the emission is localized to the treatment area. Accordingly, there is generally little far-field emission that might interfere with, for example, domestic appliances.

The devices described herein generally induce current at a much higher frequency than tissue-stimulating devices such as, for example, inductive bone-healing stimulators that pulse coils to produce a magnetic field or capacitive stimulators that produce a pulsed electric field.

Positioning of Therapeutic Devices

Therapeutic devices such as a PEMF apparatus, a transcutaneous electrical neural stimulator (TENS), or a static magnet array can be positioned at particular points on the body to achieve an enhanced medical therapeutic effect, e.g., accelerate healing, reduce pain, swelling and bruising. TENS operates by causing an electric current to be passed between electrodes placed on the skin over, for example, a painful area. Devices are described herein that can induce electrical current in a bodily tissue without the use of electrodes that are applied to the skin.

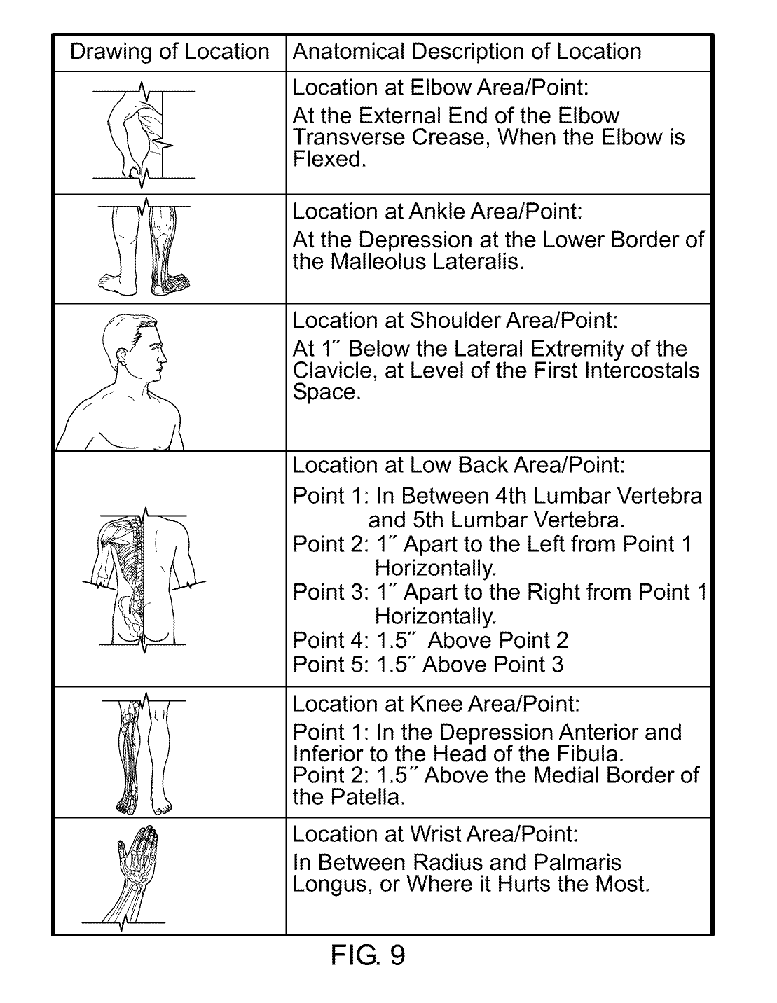

A therapeutic device can be positioned and operated at a specific acupuncture point, including but not limited to the following: the external end of the elbow transverse crease; the depression at the lower border of the malleolus lateralis; below (e.g., about 1 inch below) the lateral extremity of the clavicle at the level of the first intercostals space; between the fourth lumbar vertebra and the fifth lumbar vertebra; 1 inch to the right or left (horizontally) of the position between the fourth lumbar vertebra and the fifth lumbar vertebra; a depression anterior or inferior to the head of the fibula; about 1.5 inches above the medial border of the patella; between the radius and the palmaris longus; or at a position of pain (e.g., where the pain sensation is the strongest in an individual). FIG. 9 depicts specific anatomical locations where a therapeutic device described herein can be placed on an individual as part of a treatment program (e.g., a treatment for the reduction or elimination of pain).

The therapeutic devices described herein can be used in combination with specific acupuncture positioning techniques to reduce or eliminate pain. Examples of pain-related disorders include, for example, pain response elicited during tissue injury (e.g., inflammation, infection, and ischemia), pain associated with musculoskeletal disorders (e.g., joint pain such as that associated with arthritis, toothache, and headaches), pain associated with surgery, pain related to irritable bowel syndrome, and chest pain.

PEMF devices described herein can also be used in combination with a therapeutic substance to reduce or eliminate pain. Therapeutic substances include, for example, topical rubefacients, analgesics, anti-inflammatories, anesthetics, or a combination thereof. Rubefacients include, but are not limited to, salicylates, nicotinate esters, capsaicin, isopropanol, menthol, extracts or oils from cloves, garlic, ginger, horseradish, mustard, nettle, rosemary, or rue, or any combination thereof. The therapeutic substance may create a hot or cold sensation. In some cases, a combination of therapeutic substances may be included to create, for example, a sensation of hot and cold together. For example, capsaicin may be included to provide a hot sensation, and menthol may be included to provide a cold sensation. The electromagnetic field from the PEMF device may enhance the efficacy of the therapeutic substance, providing rapid or instant relief to a user. Relief from the therapeutic substance may be experienced by a user prior to relief from the PEMF device. A duration of efficacy of the therapeutic substance may be lengthened by the use of an extended release formulation. Typically, the duration of efficacy of the PEMF device exceeds the duration of efficacy of the therapeutic substance, such that the PEMF device provides relief after the therapeutic substance is no longer active. In an example, a therapeutic substance provides temporary pain relief, thereby masking pain before the PEMF device takes effect.

The therapeutic substance may be provided on an applicator arranged to affix a low thermal PEMF to a portion of a human body, such that the therapeutic substance contacts the body. As used herein, “low thermal PEMF device” generally refers to a PEMF device that operates at a sufficiently low field strength so as not to produce tissue heating. In some cases, the therapeutic substance may be included in an adhesive composition used to affix a PEMF device to a portion of a human body. The adhesive composition may include, for example, a hypoallergenic, non-toxic, FDA-approved pharmaceutical grade adhesive generally known in the art to adhere to a range of skin types. The adhesive composition may include one or more additional additives, such as a hydrophilic polymer for hydrating skin or wound surfaces, an antibacterial or antimicrobial substance, etc. The hydrophilic polymer may be, for example, a hydrogel. As used herein, “hydrogel” generally refers to a network of polymer chains (e.g., polyvinyl alcohol, sodium polyacrylate, acrylate polymers and copolymers, and other natural and synthetic polymers) that are hydrophilic, sometimes in the form of a colloidal gel with water as the dispersion medium.

In certain cases, the adhesive composition and the therapeutic substance may be provided to the applicator separately (e.g., in separate layers). Other layers may also be included (e.g., a hydrogel layer) in an arrangement such that the adhesive composition can adhere to bodily tissue. A release liner may be used to maintain adhesive properties of the adhesive composition before use.

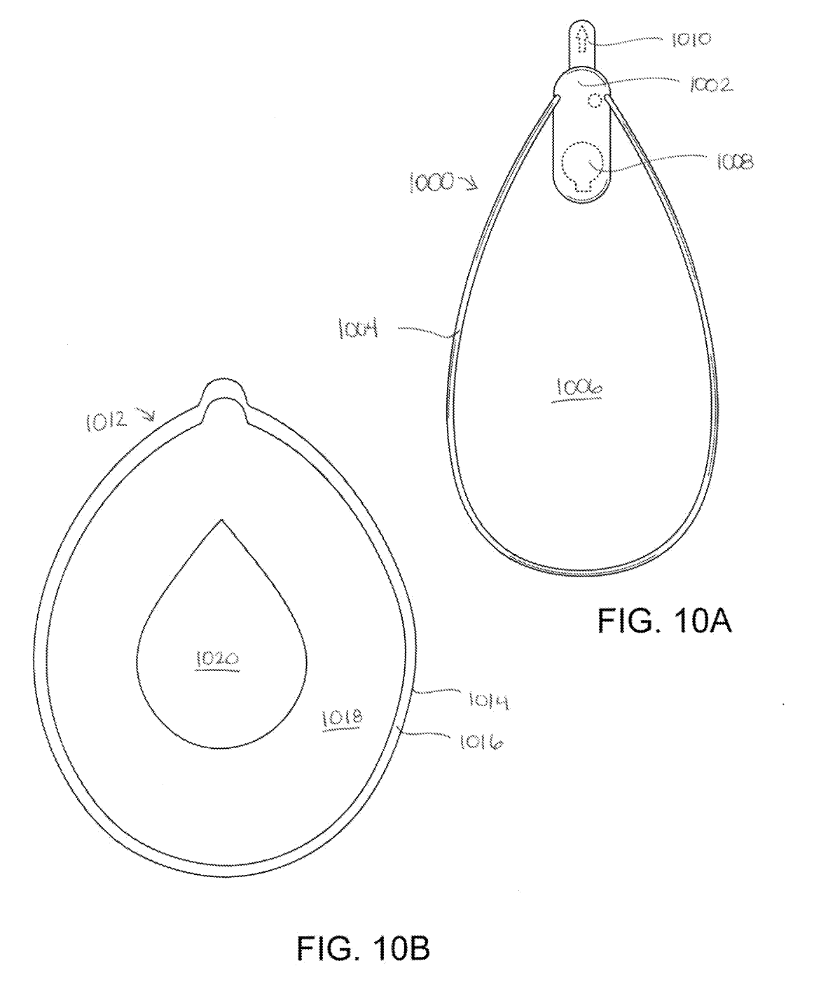

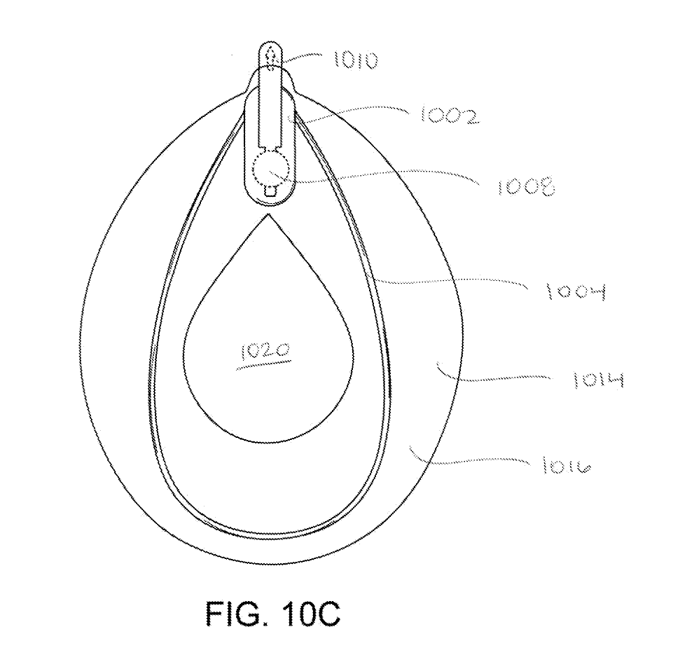

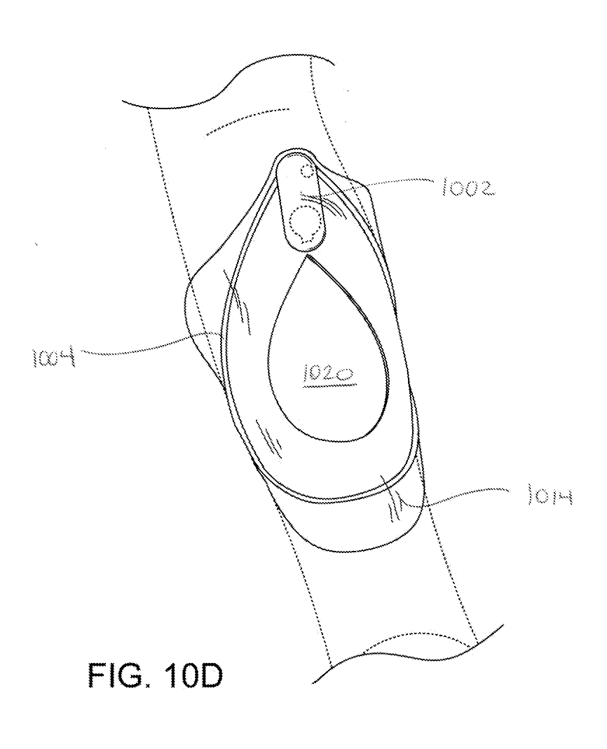

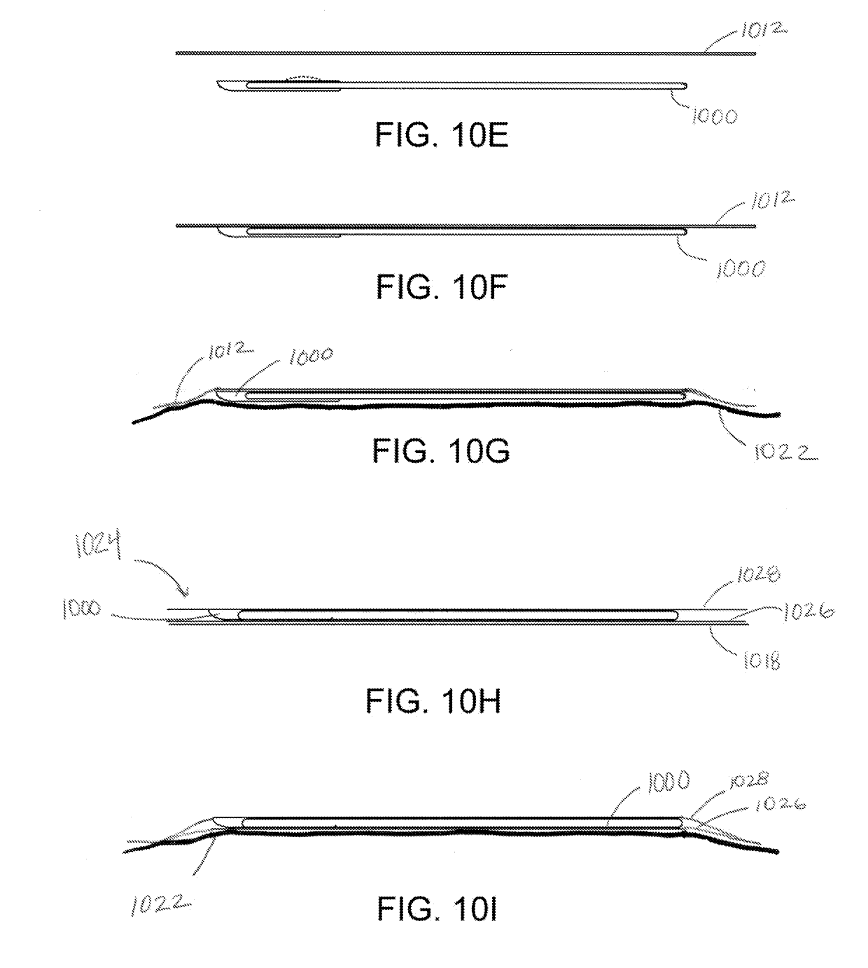

FIGS. 10A-10I, 11A-11B, 12A and 12B show various views and implementations of PEMF devices and applicators. A surface of each applicator generally supports an adhesive for adhering the PEMF device to a surface, such as an article of clothing or a human body. It is to be understood that in some cases, the applicator includes one or more therapeutic substance, one or more additives, or both, while in other cases, the applicator may not include a therapeutic substance.

FIG. 10A shows PEMF device 1000 with body 1002 and single-turn loop antenna 1004. PEMF device 1000 may be a low thermal PEMF device. Single-turn loop antenna 1004 defines opening 1006, and may have a size and geometric shape (e.g., square, circle, rectangle, oval, diamond, teardrop, etc.) designed to treat a selected region of a human body (e.g., eye, elbow, calf, shoulder, back, etc.). Body 1002 includes power source 1008 and activator 1010, as well as an electromagnetic field generator (not shown) arranged to radiate an electromagnetic field as described herein.

FIG. 10B shows applicator 1012. Applicator 1012 is shaped to overlay PEMF device 1000, thereby adhering the PEMF device to a surface. Applicator 1012 includes flexible substrate 1014, with adhesive composition 1016 as described herein applied to a first surface of the flexible substrate. The flexible substrate may be, for example, a transparent, translucent, or opaque polymeric material generally known for use as a bandage, tape, or patch. In some cases, adhesive composition 1016 includes one or more therapeutic substances. In certain cases, adhesive composition 1016 is provided to flexible substrate 1014 separately from one or more therapeutic substances (e.g., as different layers) or other additives (or layers). Release liner 1018 contacts adhesive composition 1016 on flexible substrate 1014.

FIG. 10C shows PEMF device 1000 adhered to applicator 1012 after removal of release liner 1018. PEMF device 1000 is aligned with applicator 1012 such that flexible substrate 1014 overlays antenna 1004, with the antenna adhering to the flexible substrate, and opening 1020 of the applicator aligned with opening 1006 of the PEMF device. As used herein, “align” generally means positioned such that a first opening at least partially overlaps with a second opening. Adhesive composition 1016 on flexible substrate 1014 of applicator 1012 adheres PEMF device 1000 to a surface such that antenna 1004 conforms to contours of the surface. The therapeutic substance, if present, contacts a first portion of the surface, and a second portion of the surface is exposed through opening 1020. Compared to a flexible substrate with no opening, flexible substrate 1014 with opening 1020 reduces the area of the surface covered with the flexible substrate as well as adhesive. If PEMF device 1000 is adhered to a human body, opening 1020 allows air to reach the exposed portion of the body, reducing the amount of adhesive in contact with the body and allowing the skin to breathe more easily. If PEMF device 1000 is adhered to an article of clothing, opening 1020 allows air to flow through applicator 1012 and to reach the bodily tissue underneath. Thus, opening 1020 improves comfort and wearability of PEMF device 1000.

In some cases, activator 1010 extends away from antenna 1004 as shown in FIGS. 10A and 10C. In other cases, activator 1010 extends toward opening 1006 defined by antenna 1004. Activator 1010 may be removed from PEMF device 1000 to initiate operation of the PEMF device before or after applicator 1012 is applied a surface. After activator 1010 is removed from PEMF device 1000, the radiated electromagnetic field impinges on the bodily tissue proximate the PEMF device. FIG. 10D shows PEMF device 1000 adhered to a forearm of a human body with applicator 1012, such that a first portion of the body is in contact with the applicator, and a second portion of the body is exposed to air through opening 1020. Antenna 1004 of PEMF device 1000 is in contact with bodily tissue of the forearm.

FIG. 10E shows a side view of applicator 1012, with release liner removed, aligned with PEMF device 1000 before the PEMF device is adhered to the applicator. FIG. 10F shows a side view of PEMF device 1000 adhered to applicator 1012 before application to a surface. FIG. 10G shows applicator 1012 adhering PEMF device 1000 to surface 1022, such that the PEMF device, including the antenna, conforms to the surface. As shown herein, surface 1022 is relatively planar. However, surface 1022 may be curved in one or more dimensions, with a range of curvature radius typical of portions of a human body. In some cases, (e.g., when adhesive composition 1016 does not include a therapeutic substance), surface 1022 may be an interior or exterior surface of an article of clothing. A user may wear an article of clothing with PEMF device 1000 adhered to the article of clothing proximate the portion of the body to be treated, such that electromagnetic radiation from PEMF device 1000 impinges a selected portion of the body. In some cases, the article of clothing may be close fitting or snug (e.g., made of a knit or elasticized fabric), such that antenna 1004 is held close to or conforms to the body. It may be advantageous for the article of clothing to have a thickness of less than 5 mm (e.g., between 1 mm and 3 mm) such that the electromagnetic radiation advantageously reaches bodily tissue.

FIG. 10H depicts PEMF device 1000 fixed in applicator 1024 Applicator 1024 includes first flexible substrate 1026 and second flexible substrate 1028, each defining an opening. The openings may be aligned with opening 1006 defined by antenna 1004 of PEMF device 1000, such that a common opening is formed. PEMF device 1000 is positioned (e.g., fixed, secured, or sandwiched) between first flexible substrate 1026 and second flexible substrate 1028 (e.g., with an adhesive). The exterior surface of first flexible substrate 1026 may support an adhesive composition, a therapeutic substance, one or more additives, or any combination thereof as described herein. In some cases, as shown in FIG. 10H, applicator 1024 includes release liner 1018 in contact with the adhesive composition on first flexible substrate 1026. The release liner may be removed from applicator 1024 before PEMF device 1000 is adhered to surface 1022. FIG. 10I shows applicator 1024 (and thus PEMF device 1000) adhered to surface 1022. As described herein, for a PEMF device fixed in an applicator, adhering the applicator to a surface and adhering the PEMF device to the surface are used interchangeably. That is, adhering the applicator to the surface is also understood to include adhering the PEMF device, fixed in the applicator, to the surface.

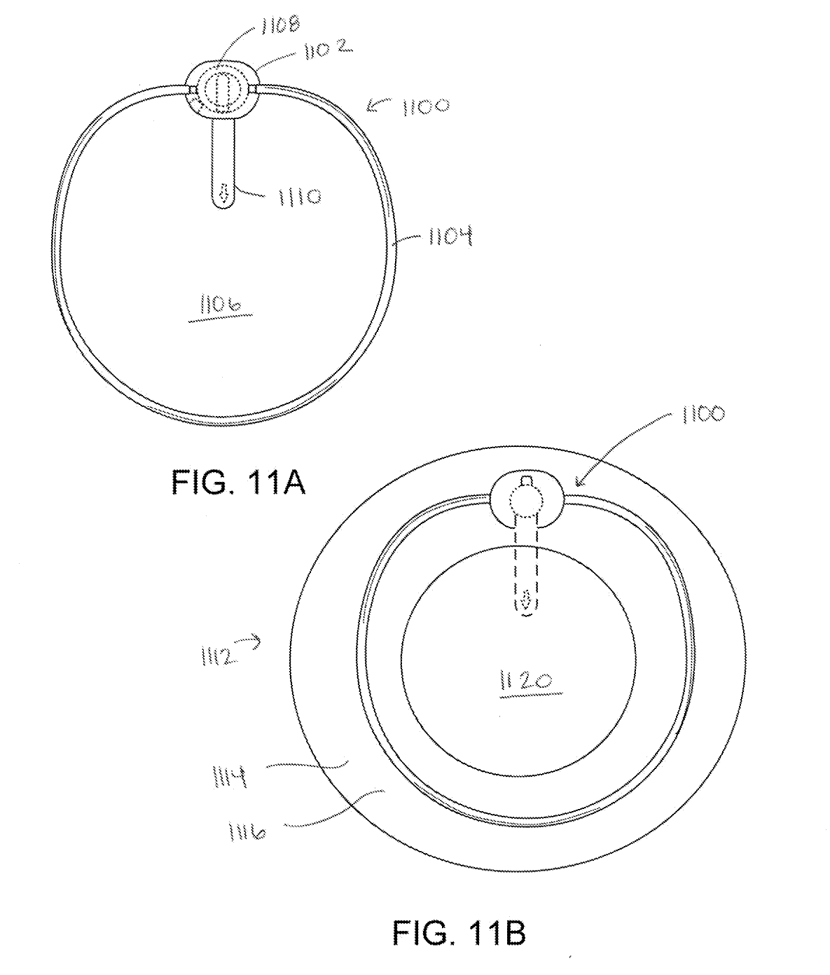

FIG. 11A depicts PEMF device 1100 with body 1102 and single-turn loop antenna 1104. PEMF device 1100 may be a low thermal PEMF device. Single-turn loop antenna 1104 defines opening 1106. Body 1102 includes power source 1108 and activator 1110, as well as an electromagnetic field generator (not shown) arranged to radiate an electromagnetic field as described herein.

FIG. 11B shows applicator 1112 shaped to overlay PEMF device 1100. Applicator 1112 includes flexible substrate 1114, with adhesive composition 1116 as described herein applied to a first surface of the flexible substrate. In some cases, adhesive composition 1116 includes one or more therapeutic substances and/or one or more additives. In certain cases, adhesive composition 1116 is provided to flexible substrate 1114 separately from one or more therapeutic substances or additives (e.g., as different layers). A release liner may be in contact with adhesive composition 1116 before PEMF device 1100 is adhered to the applicator. In some embodiments, PEMF device 1100 may be fixed between flexible layers of an applicator, as described with respect to PEMF device 1000 in FIG. 10H.

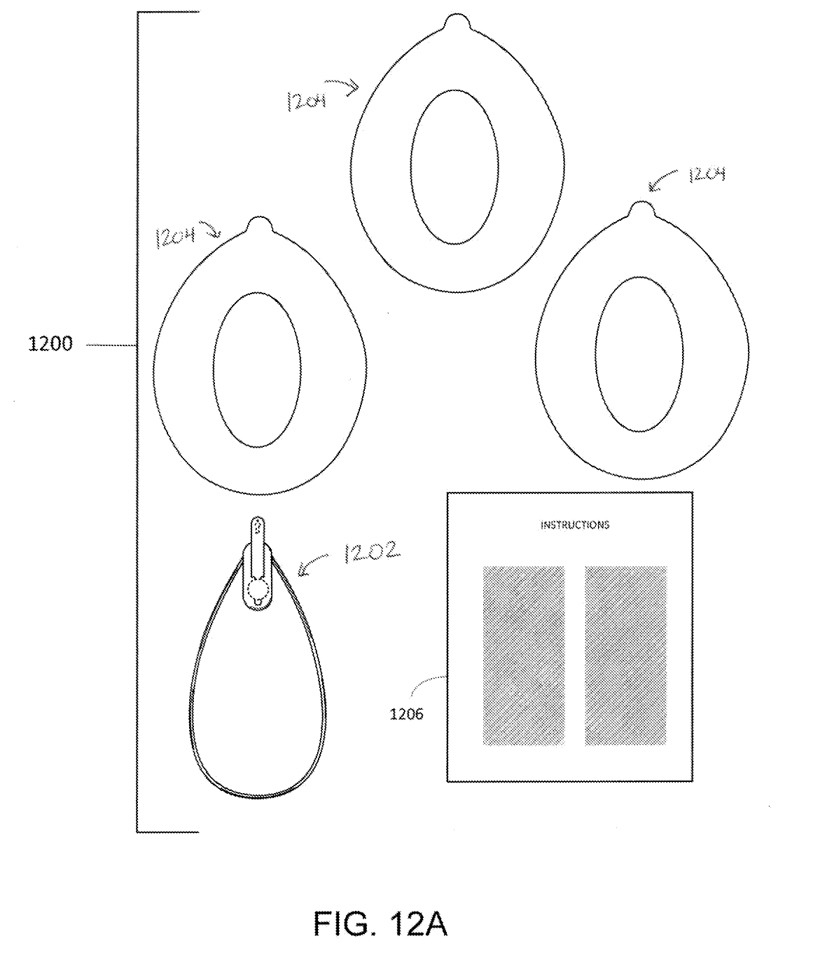

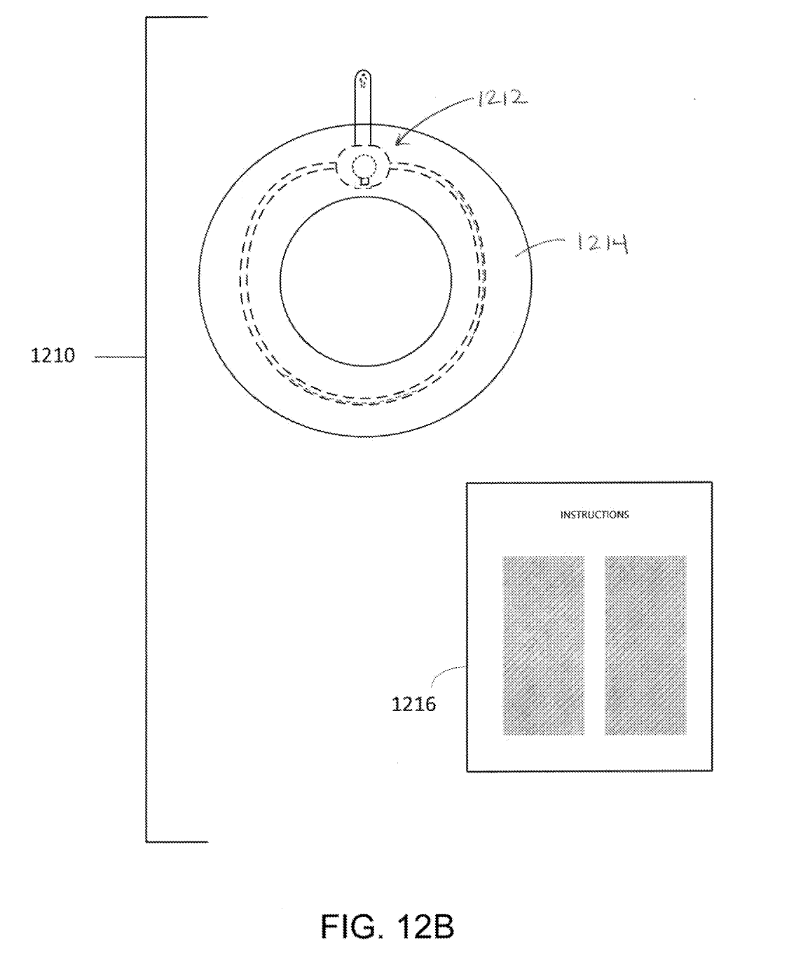

FIG. 12A depicts kit 1200 including PEMF device 1202, applicator 1204, and instructions for use 1206. Instructions 1206 may include a description of how to apply PEMF device 1202 to a surface (e.g., a human body) with applicator 1204, as described with respect to FIGS. 10A-10D. In some cases, kit 1200 includes more than one applicator 1204. In an example, kit 1200 includes four applicators 1204. A first applicator includes adhesive and a first therapeutic substance. A second applicator includes adhesive and a second therapeutic substance. A third applicator includes adhesive and a combination of the first and second therapeutic substances. A fourth applicator includes adhesive and no therapeutic substance. The therapeutic substances may be, for example, capaicincapsaicin and menthol, selected to provide sensations of hot and cold, respectively. Thus, a user may be able to select an applicator to provide a desired therapeutic effect.

FIG. 12B depicts kit 1210 including PEMF device 1212. PEMF device 1212 is fixed in applicator 1214. An exterior surface of applicator 1214 includes an adhesive composition as described with respect to FIG. 10H. The adhesive composition may include a therapeutic substance. A release liner may be in contact with the adhesive composition. Instructions for use 1214 may include a description of how to apply PEMF device 1212 to a surface (e.g., a human body) by removing the release liner and adhering the PEMF device 1212 to the surface at a desired location.

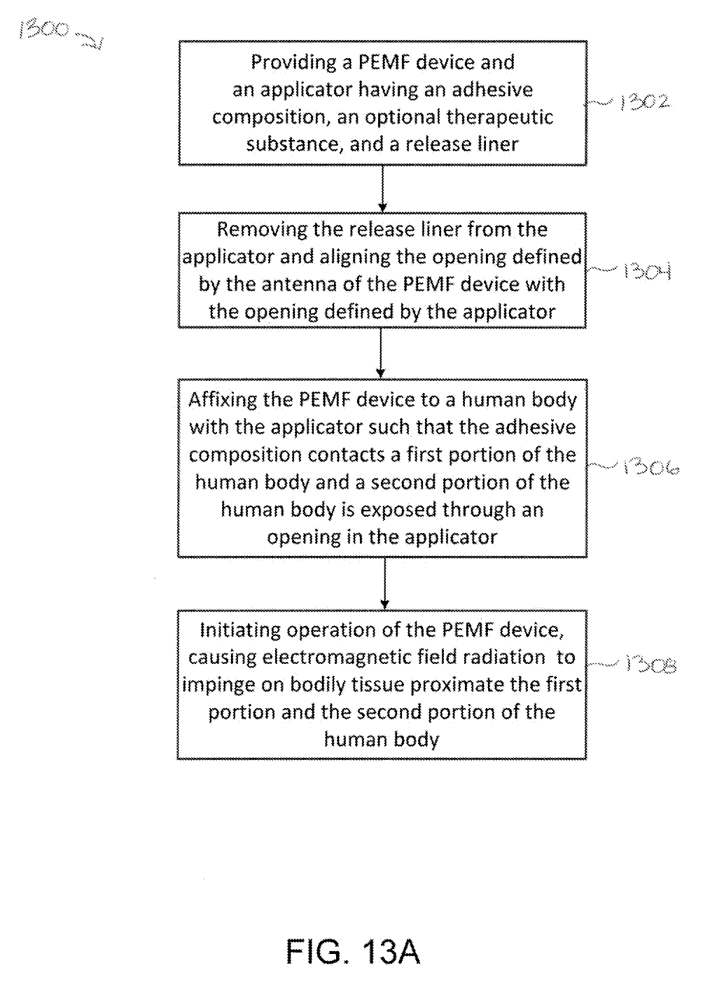

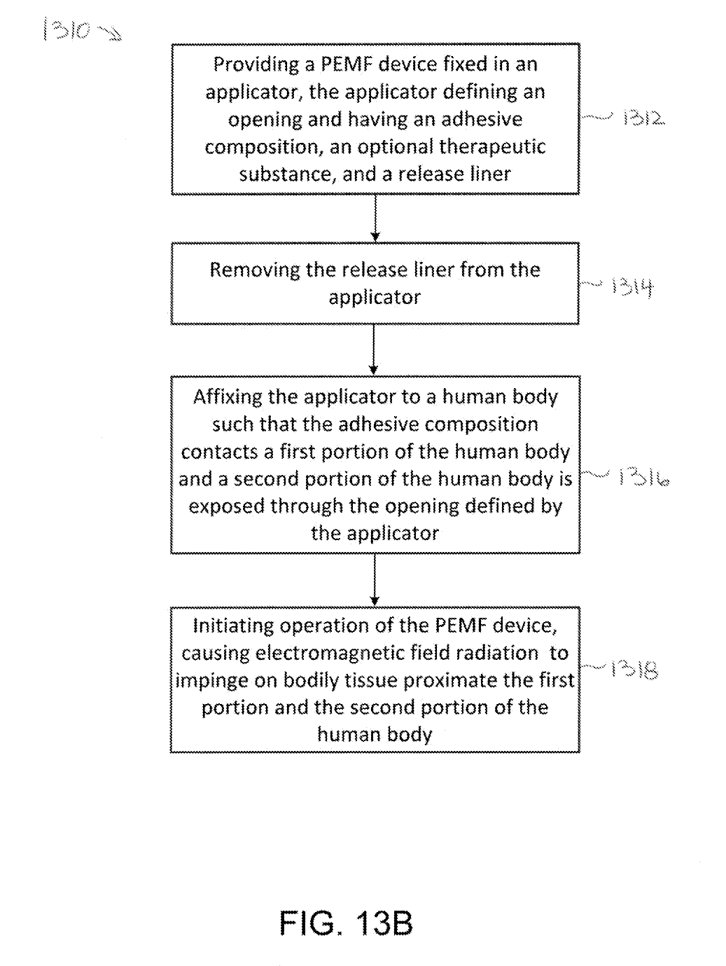

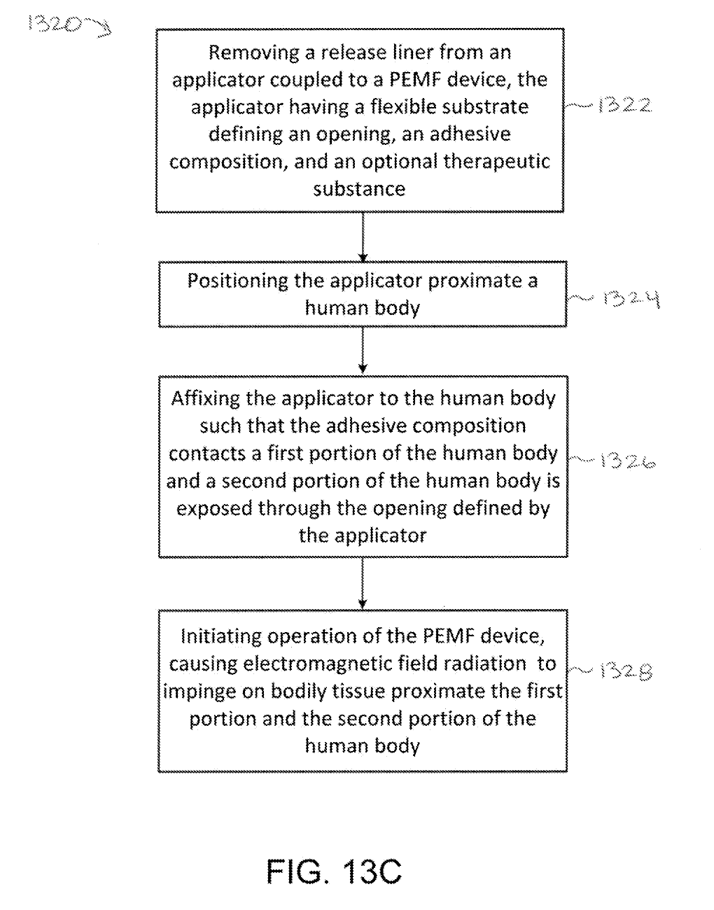

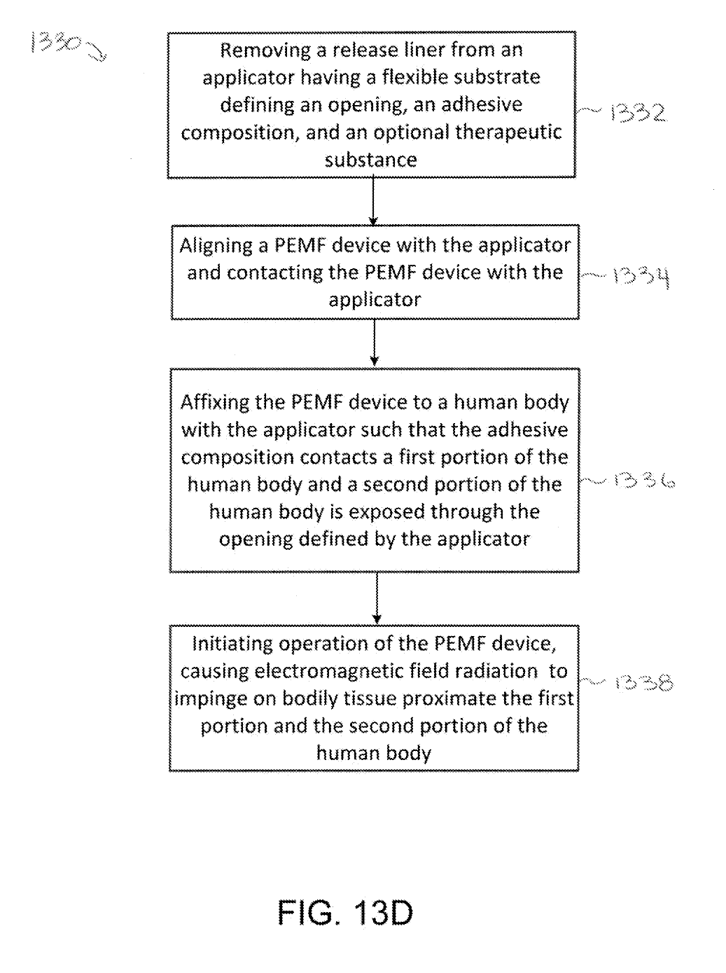

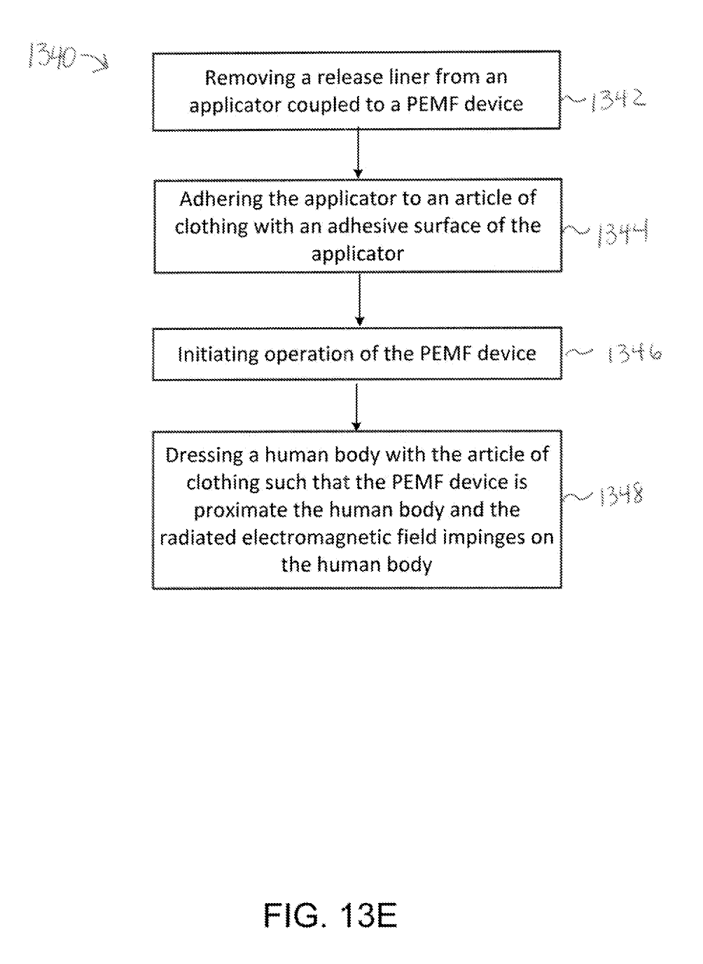

FIGS. 13A-13E are flow charts describing methods of applying a PEMF device as described herein to a surface with an adhesive applicator having a peel-off release liner. Features of the PEMF device and applicator are described herein with respect to FIGS. 10A-10H. In some cases, the order of operation in any of FIGS. 13A-13E may be altered, additional steps may be added, or steps may be omitted. A therapeutic substance may be optionally included in the adhesive composition or in a layer on the applicator proximate the adhesive composition.

FIG. 13A is a flow chart describing method 1300 of applying a PEMF device to a human body with an adhesive applicator. In 1302, a PEMF device and an applicator are provided. The applicator includes a flexible substrate as described with respect to FIGS. 10A-10H. The flexible substrate supports an adhesive composition and an optional therapeutic substance. A release liner is in contact with the adhesive composition. In 1304, the release liner is removed from (e.g., peeled off of) the applicator to expose the adhesive composition, and the PEMF device is positioned proximate the adhesive surface of the applicator such that the opening defined by the antenna is aligned with the opening defined by the applicator. In 1306, the PEMF device is affixed to a human body such that the adhesive composition (and optional therapeutic substance) contacts a first portion of the body and a second portion of the body is exposed to air through an opening in the applicator. The antenna of the PEMF device is in contact with bodily tissue. In 1308, operation of the PEMF device is initiated such that an electromagnetic field impinges on bodily tissue proximate the first and second portions of the body.

FIG. 13B is a flow chart describing method 1310 of applying a PEMF device fixed in an applicator to a human body. In 1312, a PEMF device fixed in an applicator is provided. The applicator defines an opening and has an adhesive composition and an optional therapeutic substance on one side of the applicator. A release liner is in contact with the adhesive composition. In 1314, the release liner is removed from (e.g., peeled off of) the applicator to expose the adhesive composition. In 1316, the applicator (and thus the PEMF device) is affixed to a human body such that the adhesive composition (and optional therapeutic substance) contacts a first portion of the body and a second portion of the body is exposed to air through an opening defined the applicator. The antenna of the PEMF device is typically fixed in the applicator such that it does not contact bodily tissue. In 1318, operation of the PEMF device is initiated such that an electromagnetic field impinges on bodily tissue proximate the first portion and the second portion of the body.

FIG. 13C is a flow chart describing method 1320 of applying a PEMF device fixed in an applicator to a human body. In 1322, a release liner is removed from an applicator coupled to (e.g., encasing or at least partially encasing) a PEMF device. The PEMF device may be sandwiched between two flexible layers or substrates of the applicator. In 1324, the applicator (and thus the PEMF device) is positioned proximate a human body. In 1326, the applicator is affixed to the human body such that the adhesive composition (and optional therapeutic substance) contacts a first portion of the body and a second portion of the body is exposed to air through an opening defined the applicator. The antenna of the PEMF device is fixed in the applicator and does not contact bodily tissue. In 1328, operation of the PEMF device is initiated such that an electromagnetic field impinges on bodily tissue proximate the first portion and the second portion of the body.

FIG. 13D is a flow chart describing method 1330 of applying a PEMF device to a human body with an applicator. In 1332, a release liner is removed from an applicator. In 1334, the PEMF device is aligned with the applicator and contacted with the applicator to adhere the PEMF device to the applicator such that the opening defined by the antenna is aligned with (e.g., overlaps) the opening defined by the applicator. In 1336, the PEMF device is affixed to the human body with the applicator such that the adhesive composition (and optional therapeutic substance) contacts a first portion of the body and a second portion of the body is exposed to air through an opening defined the applicator. The antenna of the PEMF device contacts the human body. In 1338, operation of the PEMF device is initiated such that an electromagnetic field impinges on bodily tissue proximate the first portion and the second portion of the body.

FIG. 13E is a flow chart describing method 1340 of applying a PEMF device fixed in an applicator to an article of clothing with an applicator. In 1342, a release liner is removed from an adhesive surface of an applicator coupled to (e.g., encasing or at least partially encasing) a PEMF device. In 1344, the applicator (and thus the PEMF device) is adhered to an article of clothing (e.g., an interior surface or an exterior surface) with the adhesive surface. Typically, the antenna of the PEMF device is fixed between layers of the applicator and does not contact the article of clothing. In 1346, operation of the PEMF device is initiated. In 1348, a human body is dressed with the article of clothing such that the PEMF device is proximate the human body, and radiated electromagnetic field impinges on the human body. In some cases, a PEMF device may be affixed to article of clothing in a process similar to that described in FIG. 13D, such that the PEMF device is contacted with an adhesive surface of the applicator, and then the adhesive surface of applicator is contacted with a surface of the article of clothing to adhere the PEMF device to the article of clothing. In this case, the antenna of the PEMF device contacts the article of clothing.

Other implementations are within the scope of the following claims.

What is claimed is:

1. A device comprising:
a pulsed electromagnetic field device comprising:
an electromagnetic field generator;
a power source coupled to the electromagnetic field generator;
a single-turn loop antenna coupled to the electromagnetic field generator arranged to radiate an electromagnetic field; and
an activator configured to initiate radiation of the electromagnetic field;
an applicator comprising:
a flexible substrate defining an opening;
an adhesive composition supported by a first surface of the flexible substrate; and
a therapeutic substance supported by the first surface of the flexible substrate,
wherein the applicator is configured to adhere the pulsed electromagnetic field device to a human body with the adhesive composition such that:
the therapeutic substance contacts a first portion of the body,
a second portion of the body is exposed to air through the opening defined by the flexible substrate; and
the radiated electromagnetic field impinges on bodily tissue proximate the first portion and the second portion of the body.

2. The device of claim 1, wherein the applicator further comprises a second flexible substrate defining a second opening and the antenna is fixed between the second flexible substrate and a second surface of the first flexible substrate such that the openings in the first flexible substrate and the second flexible substrate align to form a common opening, and wherein the applicator is configured to adhere the pulsed electromagnetic field device to the human body with the adhesive composition such that the second portion of the body is exposed to air through the common opening.

3. The device of claim 1, further comprising a release liner in contact with the adhesive composition.

4. The device of claim 1, wherein the adhesive composition is non-toxic.

5. The device of claim 1, further comprising a hydrogel supported by the one side of the flexible substrate.

6. The device of claim 1, wherein the adhesive composition comprises the hydrogel.

7. The device of claim 1, wherein the therapeutic substance is a topical rubefacient.

8. The device of claim 7, wherein the topical rubefacient comprises a salicylate, a nicotinate ester, capsaicin, isopropanol, menthol, or a combination thereof.

9. The device of claim 7, wherein the topical rubefacient comprises an extract or oil of cloves, garlic, ginger, horseradish, mustard, nettle, rosemary, rue, or a combination thereof.

10. The device of claim 1 wherein the therapeutic substance has analgesic properties, anti-inflammatory properties, anesthetic properties, or a combination thereof.

11. The device of claim 1, wherein the adhesive composition comprises the therapeutic substance.

12. The device of claim 1, wherein the duration of the electromagnetic field radiation exceeds the duration of efficacy of the therapeutic substance.

13. A kit comprising:
a pulsed electromagnetic field device comprising:
an electromagnetic field generator;
a power source coupled to the electromagnetic field generator;
a single-turn loop antenna coupled to the electromagnetic field generator and arranged to radiate an electromagnetic field; and
an activator configured to initiate radiation of the electromagnetic field;
an applicator comprising:
a flexible substrate defining an opening; and
an adhesive composition and a therapeutic substance supported by a first surface of the flexible substrate; and
a release liner in contact with the adhesive composition; and
instructions for adhering the pulsed electromagnetic field device to a human body with the applicator such that:
the therapeutic substance contacts a first portion of the body,
a second portion of the body is exposed to air through the opening defined by the flexible substrate,
radiation of the electromagnetic field is initiated, and
the radiated electromagnetic field impinges on bodily tissue proximate the first portion and the second portion of the body.

14. The kit of claim 13, wherein the applicator further comprises a second flexible substrate defining a second opening, and the antenna is fixed between the second flexible substrate a second surface of the first flexible substrate such that the openings in the first flexible substrate and the second flexible substrate align to form a common opening, and wherein the applicator is configured to adhere the pulsed electromagnetic field device to the human body with the adhesive composition such that the second portion of the body is exposed to air through the common opening.

15. A method comprising
providing a pulsed electromagnetic field device and an applicator, the applicator comprising:
a flexible substrate defining an opening;
an adhesive composition supported by a first surface of the flexible substrate;
a therapeutic substance supported by the first surface of the flexible substrate; and
a release liner in contact with the adhesive composition;
removing the release liner from the adhesive composition;
aligning the opening defined by the flexible substrate with an opening defined by the pulsed electromagnetic field device; and
contacting the pulsed electromagnetic field device with the adhesive composition supported by the first surface of the flexible substrate;
affixing the pulsed electromagnetic field device to a human body with the applicator such that the therapeutic substance contacts a first portion of the human body and a second portion of the human body is exposed to air through the opening defined by the flexible substrate; and
causing the pulsed electromagnetic field device to radiate an electromagnetic field that impinges on the human body.

16. The method of claim 15, wherein the duration of radiation of the electromagnetic field exceeds the duration of efficacy of the therapeutic substance.

17. A method comprising
providing a pulsed electromagnetic field device fixed in an applicator, the applicator defining an opening and comprising:
an adhesive composition supported by a first surface of the applicator;
a therapeutic substance supported by the first surface of the applicator; and
a release liner in contact with the adhesive composition;
removing the release liner from the adhesive composition;
affixing the applicator to a human body such that the therapeutic substance contacts a first portion of the human body and a second portion of the human body is exposed to air through the opening defined by the applicator; and
causing the pulsed electromagnetic field device to radiate an electromagnetic field that impinges on the human body.

18. A method comprising
positioning a pulsed electromagnetic field device and an applicator proximate a human body, the applicator comprising:
a flexible substrate defining an opening;
an adhesive composition supported by a first surface of the flexible substrate; and
a therapeutic substance supported by the first surface of the flexible substrate; and
affixing the applicator to the human body such that the therapeutic substance contacts a first portion of the human body and a second portion of the human body is exposed to air through the opening defined by the flexible substrate; and
causing the pulsed electromagnetic field device to radiate an electromagnetic field that impinges on the human body.

19. A method comprising
positioning a pulsed electromagnetic field device fixed in an applicator proximate a human body, the applicator comprising:
a flexible substrate defining an opening;
an adhesive composition supported by a first surface of the flexible substrate; and
a therapeutic substance supported by the first surface of the flexible substrate; and
affixing the applicator to the human body such that the therapeutic substance contacts a first portion of the human body and a second portion of the human body is exposed to air through the opening defined by the flexible substrate; and
causing the pulsed electromagnetic field device to radiate an electromagnetic field that impinges on the human body.

20. A device comprising:
a pulsed electromagnetic field device comprising:
an electromagnetic field generator;
a power source coupled to the electromagnetic field generator;
a single-turn loop antenna coupled to the electromagnetic field generator arranged to radiate an electromagnetic field; and
an activator configured to initiate radiation of the electromagnetic field;
an applicator comprising:
a flexible substrate defining an opening; and
an adhesive composition supported by a first surface of the flexible substrate;
wherein the applicator is configured to adhere the pulsed electromagnetic field device to a surface with the adhesive composition such that a portion of the surface is exposed to air through the opening defined by the flexible substrate.

21. The device of claim 20, wherein the applicator further comprises a second flexible substrate defining a second opening, the antenna is fixed between the second flexible substrate a second surface of the first flexible substrate such that the openings in the first flexible substrate and the second flexible substrate align to form a common opening, and wherein the applicator is configured to adhere the pulsed electromagnetic field device to the surface with the adhesive composition such that the portion of the surface is exposed to air through the common opening.

22. The device of claim 20, further comprising a release liner in contact with the adhesive composition.

23. The device of claim 20, wherein the surface is an article of clothing.

24. The device of claim 20, wherein the surface is bodily tissue.

25. A method comprising:
adhering a pulsed electromagnetic field device to an article of clothing with an applicator;
causing the pulsed electromagnetic field device to radiate an electromagnetic field; and
dressing a human body with the article of clothing, such that the pulsed electromagnetic field device is proximate the human body and the radiated electromagnetic field impinges on the human body.
